SEQUENCE LISTING (16 sequences)

SEQ ID NO: 1 - DNA, 13 bp, Artificial Sequence
  HLA-B*1502 forward primer
atggcgccccggg

SEQ ID NO: 2 - DNA, 19 bp, Artificial Sequence
  HLA-B*1502 reverse primer
tagtagccgcgcaggttcc

SEQ ID NO: 3 - DNA, 19 bp, Artificial Sequence
  HLA-B*1502 probe 1
aacacacagatctacaagg

SEQ ID NO: 4 - DNA, 19 bp, Artificial Sequence
  HLA-B*1502 probe 2
aacacacagatctccaaga

SEQ ID NO: 5 - DNA, 17 bp, Artificial Sequence
  HLA-B*3802 forward primer
gccgcgagtccgagaga

SEQ ID NO: 6 - DNA, 20 bp, Artificial Sequence
  HLA-B*3802 reverse primer
gtgcgcaggttctctcggta

SEQ ID NO: 7 - DNA, 15 bp, Artificial Sequence
  HLA-B*3802 probe 1
ccggagtattgggac

SEQ ID NO: 8 - DNA, 15 bp, Artificial Sequence
  HLA-B*3802 probe 2
ccggaatattgggac

SEQ ID NO: 9 - DNA, 17 bp, Artificial Sequence
  HLA-B*1301 forward primer
agccccgcttcatcacc

SEQ ID NO: 10 - DNA, 20 bp, Artificial Sequence
  HLA-B*1301 reverse primer
tccttgccgtcgtaggctaa

SEQ ID NO: 11 - DNA, 17 bp, Artificial Sequence
  HLA-B*1301 probe 1
cacatcatccagaggat

SEQ ID NO: 12 - DNA, 16 bp, Artificial Sequence
  HLA-B*1301 probe 2
acacttggcagacgat

SEQ ID NO: 13 - DNA, 19 bp, Artificial Sequence
  HLA-B*3901 forward primer
gcgagtccgagagaggagc

SEQ ID NO: 14 - DNA, 19 bp, Artificial Sequence
  HLA-B*3901 reverse primer
tagtagccgcgcaggttcc

SEQ ID NO: 15 - DNA, 17 bp, Artificial Sequence
  HLA-B*3901 probe 1
tccaattcacagactga

SEQ ID NO: 16 - DNA, 15 bp, Artificial Sequence
  HLA-B*3901 probe 2
caacacacagactga

(19) United States
(12) Patent Application Publication
(10) Pub. No.: US 2022/0259655 A1
(43) Pub. Date: Aug. 18, 2022

(54) METHODS FOR ASSESSING THE RISK OF DEVELOPING SEVERE CUTANEOUS ADVERSE DRUG REACTIONS INDUCED BY DISEASE-MODIFYING ANTIRHEUMATIC DRUGS, DETECTION KIT THEREOF AND USES THEREOF

(71) Applicant: CHANG GUNG MEMORIAL HOSPITAL, LINKOU, Taoyuan (TW)

(72) Inventors: Wen-Hung CHUNG, Taoyuan (TW); Shuen-Iu HUNG, Taoyuan (TW); Chuang-Wei WANG, Taipei (TW)

(21) Appl. No.: 17/604,847

(22) PCT Filed: May 6, 2019
(86) PCT No.: PCT/CN2019/085639

§ 371 Date: Oct. 19, 2021

(51) Int. Cl. C12Q 1/6883; C12Q 1/6881

(52) U.S. Cl. CPC C12Q 1/6883; C12Q 2600/118; C12Q 1/6881

(57) ABSTRACT

A method for assessing the risk of severe cutaneous adverse drug reactions (SCARs) induced by disease-modifying antirheumatic drugs is provided, wherein the severe cutaneous adverse drug reactions comprises but not being limited to: Stevens-Johnson Syndrome (SJS), toxic epidermal necrolysis (TEN) and drug rash with eosinophilia and systemic symptoms (DRESS). Also provided is a detection kit for assessing the risk of developing cutaneous adverse drug reactions in a subject, said kit comprising a reagent for determining specific HLA alleles and a use of the detection kit in assessing the risk of developing cutaneous adverse drug reactions in a subject.

### TECHNICAL FIELD

The present invention provides a method for assessing the risk of developing cutaneous adverse drug reactions induced by disease-modifying anti-rheumatic drugs, especially Sulfasalazine, Mesalazine, Sulfapyridine, Olsalazine for inducing cutaneous adverse drug reactions.

### BACKGROUND

Cutaneous Adverse Drug Reactions (CADRs) have always been a major clinical problem with very diverse manifestations, ranging from mild papules (maculopapular eruption, MPE), fixed drug rash (FDE) to severe cutaneous adverse drug reactions (SCARs), which includes drug rash with eosinophilia and systemic symptoms (DRESS), Stevens Johnson Syndrome (SJS) and toxic epidermal necrolysis (TEN), and so on. The symptoms prior to the onset of Stevenson-Johnson Syndrome (SJS) and Toxic Epidermal Necrosis (TEN) are flu-like symptoms, including fever, sore throat, swollen lips, etc., which rapidly progressed to generalized erythema, blisters, and inflammation and ulceration of the mucous membranes of eyes, oral cavity and genitals. In severe cases, the symptoms are similar to those of whole body burn. The major difference between SJS and TEN is the percentage of epidermal separation: in SJS, the separation is less than 10% of the body surface area and in TEN, the separation exceeds 30% of the body surface area. The main clinical features of drug eruption with eosinophilia and systemic symptoms (DRESS) include fever, skin rash, an increase in eosinophils in the blood, lymphadenopathy and internal organ invasion. The most common and severely affected organ is the liver, which may lead to fulminant hepatitis, the most common cause of death in these patients. Other organ involvement leads to nephritis, myocarditis, pneumonia, and thyroiditis.

Adverse drug reactions are often associated with immune reactions, but the immune mechanism is extremely complicated. For example, HLA-A has about 300 subtypes and HLA-B has about 600 genotypes. Therefore, it is difficult to ascertain the immune mechanism that underlines the adverse drug reactions.

The disease-modifying antirheumatic drug Sulfasalazine (C₁₈H₁₄N₄O₅S, Formula I, trade name is Salazine, Salazopyrin® or Azulfidine®) modulates the immune system with an anti-inflammatory effect. It was approved by the US Food and Drug Administration (FDA) in 1950 for treating inflammatory bowel diseases and various inflammatory arthritis, such as: rheumatoid arthritis, ankylosing spondylitis, psoriatic arthritis and juvenile chronic arthritis and so on. Sulfasalazine and its metabolites, such as mesalazine (Mesalazine, C₇H₇NO₃, formula II) and sulfapyridine (C₁₁H₁₁N₃O₂S, formula III), and the dimer of mesalazine (Olsalazine, C14H10N2O6, C₁₄H₁₀N₂O₆, Formula IV) have anti-inflammatory, immunosuppressive and antibacterial effects. When used in the treatment of inflammatory arthritis, not only can they reduce joint pain and swelling, but they also reduce the chance of permanent joint damage and disability.

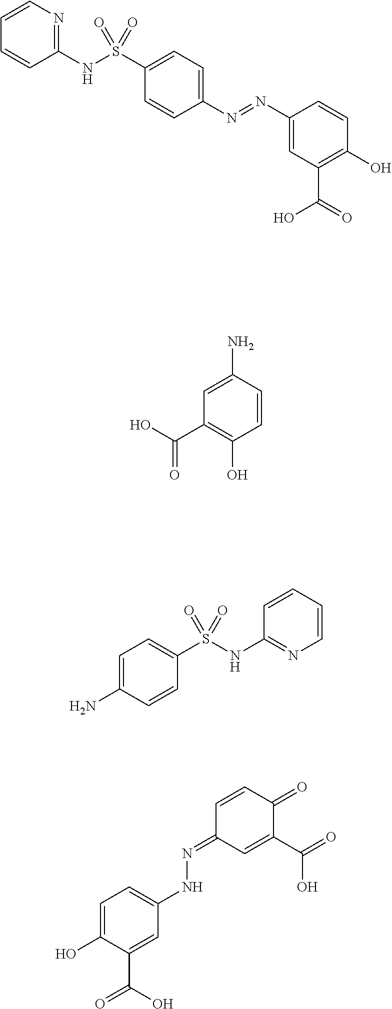 [SMILES: O=C(O)c1cc(/N=N/c2ccc(S(=O)(=O)Nc3ccccn3)cc2)ccc1O ; Nc1ccc(O)c(C(=O)O)c1 ; Nc1ccc(S(=O)(=O)Nc2ccccn2)cc1 ; O=C(O)C1=C/C(=N\Nc2ccc(O)c(C(=O)O)c2)C=CC1=O]

Although disease-modifying antirheumatic drugs can be used to treat a wide range of inflammatory diseases, their use is limited due to the higher incidence of adverse reactions in clinical setting. Approximately 25% of patients taking sulfasalazine will develop obvious side effects, including: loss of appetite, nausea, headache, neutropenia, liver problems, kidney problems, and cutaneous adverse drug reactions (CADRs), among which the cutaneous adverse drug reactions is the second most common adverse reaction. Therefore, there is still a need for assessing the risk of developing severe cutaneous adverse drug reactions (including: SJS, TEN, and DRESS) caused by disease-modifying antirheumatic drugs. The present invention addresses this need.

### SUMMARY OF THE INVENTION

The present invention provides a method for assessing the risk developing severe cutaneous adverse drug reactions (SCARs) induced by disease-modifying antirheumatic drugs in a patient. The severe cutaneous adverse drug reactions comprises Stevens Johnson Syndrome (SJS), toxic epidermal necrolysis (TEN) or drug rash with eosinophilia and systemic symptoms (DRESS). HLA-B*1502 allele, HLA-B*3802 allele or a combination thereof, a combination of HLA-B*1301 allele and HLA-B*3901 allele are associated with severe cutaneous adverse drug reactions induced by disease-modifying antirheumatic drugs.

Specifically, the present invention provides a method for assessing the risk of developing severe cutaneous adverse drug reactions induced by a disease-modifying antirheumatic drugs, comprising the step of detecting the presence of at least one allele selected from: HLA-B*1502 allele, HLA-B*3802 allele, or a combination of HLA-B*1301 and HLA-B*3901, wherein the presence of at least one allele indicates the risk of severe cutaneous adverse drug reaction. In a specific example, the drug is disease-modifying anti-rheumatic drugs (DMARDs). Disease-modifying antirheumatic drugs include (but are not limited to) Sulfasalazine, Mesalazine, Sulfapyridine or Olsalazine. Severe cutaneous adverse drug reactions include at least one adverse reaction selected from the following: Stevens Johnson Syndrome (SJS), toxic epidermal necrolysis (TEN) or drug rash with eosinophilia and systemic symptoms (DRESS). In one embodiment, the subject carries the HLA-B*1502 allele. In one embodiment, the subject carries the HLA-B*3802 allele. In one embodiment, the subject carries a combination of HLA-B*1502 allele and the HLA-B*3802 allele. In one embodiment, the subject carries a combination of HLA-B*1301 allele, HLA-B*3802 allele and HLA-B*3901 allele. In one embodiment, the subject carries a combination of HLA-B*1301 allele, HLA-B*1502 allele, the HLA-B*3802 allele and the HLA-B*3901 allele.

The present invention provides a reagent for detecting HLA-B*1502 allele, HLA-B*3802 allele, or a combination of HLA-B*3901 allele and the HLA-B*1301 allele in the manufacture of a detection kit to evaluate the risk of developing a severer cutaneous adverse drug reaction induced by a disease-modifying antirheumatic drug. The kit includes a reagent for detecting at least one allele selected from: HLA-B*1502 allele, HLA-B*3802 allele or a combination of HLA-B*1301 allele and HLA- B*3901 allele.

The presence of HLA-B*1502 allele, HLA-B*3802 allele, a combination of HLA-B*1301 allele and HLA-B*3901 allele, a combination of HLA-B*1502 allele and HLA-B*3802 allele, a combination of HLA-B*1301 allele, HLA-B*3802 allele and HLA-B*3901 allele or a combination of HLA-B*1301 allele, HLA-B*1502 allele, HLA-B*3802 allele and HLA-B*3901 allele in a subject indicates that the subject has a higher than one time, two times, three times, four times, five times, six times, seven times, eight times, nine times, ten times, eleven times, twelve times, thirteen times, fourteen times, fifteen times, sixteen times, seventeen times, eighteen times, nineteen times, 20 times , 30 times, 40 times, 50 times or higher than one time to 14 times risk of developing adverse drug reactions compared to a subject without HLA-B*1502 allele, HLA-B*3802 allele, a combination of HLA-B*1301 allele and HLA-B*3901 allele, a combination of HLA-B*1502 allele and HLA-B*3802 allele, a combination of HLA-B*1301 allele, HLA-B*3802 allele and HLA- B*3901 allele or a combination of HLA-B*1301 allele, HLA-B*1502 allele, HLA-B*3802 allele and HLA-B*3901 allele.

Any known methods in the art for detecting alleles can be used, such as (but not limited to): an oligonucleotide that specifically hybridizes to the allele, serotyping or microcytotoxicity method to determine cDNA, RNA or protein product of the allele. [Kenneth D.McClatchey.Clinical Laboratory Medicine. 2002]. In one embodiment, the oligonucleotide specifically hybridizes to the DNA of the peripheral blood of the subject. The oligonucleotide is designed for the most variable sequences of HLA-B*1301 allele and/or HLA-B*1502 allele and/or HLA-B*3802 allele and/or HLA-B*3901 allele. In one embodiment, oligonucleotide sequence of the forward primer for detecting the presence of HLA-B*1502 is 5′-ATGGCGCCCCGGG-3′ (SEQ ID No.1), the sequence of the reverse primer for detecting the presence of HLA-B*1502 is 5′-TAGTAGCCGCGCAGGTTCC-3′ (SEQ ID No. 2), the sequence of probe 1 for detecting the presence of HLA-B*1502 is 5′-AACACACAGATCTACAAGG-3′ (SEQ ID No. 3) and sequence of probe 2 for detecting the presence of HLA-B*1502 is 5′-AACACACAGATCTCCAAGA-3′ (SEQ ID No. 4). In a specific embodiment, the oligonucleotide sequence of the forward primer for detecting the presence of HLA-B*3802 is 5′-GCCGCGAGTCCGAGAGA-3′ (SEQ ID No.5), the sequence of the reverse primer for detecting the presence of HLA-B*3802 is 5′-GTGCGCAGGTTCTCTCGGTA-3′ (SEQ ID No. 6), the sequence of probe 1 for detecting the presence of HLA-B*3802 is 5′-CCGGAGTATTGGGAC-3′ (SEQ ID No. 7) and the sequence of probe 2 for detecting the presence of HLA-B*3802 sequence is 5′-CCGGAATATTGGGAC-3′ (SEQ ID No. 8). In another specific embodiment, oligonucleotide sequence of the forward primer for detecting the presence of HLA-B*1301 is 5′-AGCCCCGCTTCATCACC-3′ (SEQ ID No. 9), the sequence of the reverse primer for detecting the presence of HLA-B*1301 is 5′-TCCTTGCCGTCGTAGGCTAA-3′ (SEQ ID No.10), the sequence of probe 1 for detecting the presence of HLA-B*1301 is 5′-CACATCATCCAGAGGAT-3′ (SEQ ID No.11) and the sequence of probe 2 for detecting the presence of HLA-B*1301 is 5′-ACACTTGGCAGACGAT-3′ (SEQ ID No.12). In another specific embodiment, the oligonucleotide sequence of the forward primer for detecting the presence of HLA-B*3901 is 5′-GCGAGTCCGAGAGAGGAGC-3′ (SEQ ID No. 13), the sequence of the reverse primer for detecting the presence of HLA-B*3901 is 5′-TAGTAGCCGCGCAGGTTCC-3′ (SEQ ID No.14), the sequence of probe 1 for detecting the presence of HLA-B*3901 is 5′-TCCAATTCACAGACTGA-3′ (SEQ ID No.15) and the sequence of probe 2 for detecting the presence of HLA-B*3901 is 5′-CAACACACAGACTGA-3′ (SEQ ID No.16).

The present invention provides a detection kit for assessing the risk of developing severe cutaneous adverse drug reactions caused by disease-modifying antirheumatic drugs. The detection kit comprises a reagent that can detect at least one allele selected from the following: HLA-B* 1502 allele; HLA-B*3802 allele or a combination of HLA-B*1301 allele and HLA-B*3901 allele, wherein the presence of at least one allele indicates an increased risk of developing severe cutaneous adverse drug reactions caused by disease-modifying antirheumatic drugs in a subject compared to a subject without the corresponding allele. In a specific embodiment, the severe cutaneous adverse drug reaction comprises at least one adverse reaction selected from the following: Stevenson-Jonson Syndrome, toxic epidermal necrosis or drug eruption with eosinophilia and systemic symptoms.

The present invention provides methods for reducing the incidence of severe cutaneous adverse drug reactions caused by disease-modifying antirheumatic drugs or methods of treating said severe cutaneous adverse drug reactions.

The present invention also provides a method for assessing the risk of developing adverse drug reactions caused by disease-modifying antirheumatic drugs and treating said adverse drug reactions, comprising the following steps: (a) detecting at least one allele selected from the following alleles in a sample of a subject: HLA -B*1502 allele, HLA-B*3802 allele or a combination of HLA-B*1301 allele and HLA-B*3901 allele, (b) the presence of at least one of the following alleles in the sample: HLA-B*1502 allele, HLA-B* 3802 allele or the combination of HLA-B*1301 allele and HLA-B*3901 allele indicates the subject has adverse drug reactions induced by disease-modifying antirheumatic drugs; and (c) administer a drug to treat the adverse drug reaction.

In a specific embodiment, the method of treating the adverse drug reactions is administering a drug including (but not limited to) liquid, steroid, immunoglobulin, cyclosporine, anti-TNF-α agent or plasma replacement.

The present invention also relates to a method for assessing the risk of developing an adverse drug reactions induced by disease-modifying antirheumatic drugs and reducing the incidence of said adverse drug reactions, comprising the following steps: (a) detecting at least one allele selected from the following alleles in a sample of a subject : HLA-B*1502 allele, HLA-B*3802 allele or a combination of HLA-B*1301 allele and HLA-B*3901 allele, (b) the presence of at least one of the following alleles in the sample: HLA-B*1502 allele, HLA-B* 3802 allele or the combination of HLA-B*1301 allele and HLA-B*3901 allele, indicates that the subject has an increased risk of developing an adverse drug reaction and (c) the subject is not given the disease-modulating anti-rheumatic drugs.

The terms “invention” and “present invention” as used in the present invention are intended to broadly refer to the application the claims. The statements containing these terms are to be understood as not limiting the scope of the application or the scope of the claims. The working examples of the invention are defined by the application and not by the content of the present invention. This summary is a high-level overview of various aspects of the invention and is a description of some concepts that are further described in the section below. This Summary is not intended to identify key or essential features of the claimed application, and is not intended to be used solely to determine the scope of the claimed application. The objectives of the application should be understood by reference to any or all of the figures and the appropriate parts of each claim.

### WORKING EXAMPLE

In the following working example, 32 patients (including 11 SJS/TEN and 21 DRESS patients) with disease-modifying antirheumatic drug (Sulfasalazine) induced severe cutaneous adverse drug reaction were enrolled for HLA typing and the HLA typing results were compared with that of 941 normal healthy controls. The results show that HLA-B*1301 allele, HLA-B*1502 allele, HLA-B*3802, HLA-B*3901, a combination of HLA-B*1301 allele and HLA-B*3901 allele , a combination of HLA-B*1502 allele and HLA-B*3802 allele, a combination of HLA-B*1301 allele, HLA-B*3802 allele and HLA-B*3901 allele or a combination of HLA-B*1301 allele, HLA-B*1502 allele, HLA-B*3802 allele and HLA-B*3901 allele were associated with sulfasalazine induced severe cutaneous adverse drug reaction (see Table 1).

With respect to the HLA-B*1301 allele, 8 of 21 patients with sulfasalazine induced DRESS carried this genotype (38.10%), whereas only 114 of the 941 normal healthy subjects in the control group carried this genotype (12.11%). This shows the HLA-B*1301 allele is associated with sulfasalazine induced DRESS (DRESS vs. healthy control group: P=2.59×10⁻³, odds ratio or OR)=4.5 (1.8-11.0)), sensitivity: 38.10%, specificity: 87.89%). With respect to the HLA-B*3802 allele, 6 out of 11 patients with sulfasalazine induced SJS/TEN carried this genotype (54.54%), whereas only 71 out of 941 normal healthy control group (General population) carried this genotype. This shows the association of HLA-B*3802 allele with SJS/TEN induced by sulfasalazine (SJS/TEN vs. healthy control group: P=7.72×10⁻⁵, odds ratio or OR)=14.7 (4.4-49.4), sensitivity: 54.54%, specificity: 92.45%).

Further analysis of the HLA-B*1301 allele and HLA-B*3901 allele combination shows that such combination significantly increases the correlation with and sensitivity in predicting the risk of developing sulfasalazine induced DRESS (DRESS vs. healthy control group: P=4.27×10⁻⁸, odds ratio=13.1 (5.0-34.2), sensitivity: 71.43%, specificity: 83.95%).

With respect to the HLA-B*1502 allele, 5 out of 11 patients with sulfasalazine induced SJS/TEN carried this genotype (45.45%), and only 87 out of 941 normal healthy subjects in the control group carried this genotype (9.25%). This shows HLA-B*1502 allele is associated with sulfasalazine induced SJS/TEN (SJS/TEN vs. healthy control group: P=2.19×10⁻³, odds ratio=8.2 (2.4-27.4), sensitivity: 45.45%, specificity: 90.75%).

With respect to the HLA-B*3901 allele, 8 of 21 patients with sulfasalazine induced DRESS carried this genotype (38.10%) whereas only 43 of 941 normal healthy subjects in the control group carried this genotype (4.57%). This shows HLA-B*3901 is associated with sulfasalazine induced DRESS (DRESS vs. healthy control group: P=4.30×10⁻⁶, odds ratio or OR=12.2 (4.6-32.5), sensitivity: 38.10%, specificity: 95.43%).

Further analysis of the HLA-B*1502 allele and HLA-B*3802 allele combination shows that such combination significantly increases the correlation with and sensitivity in predicting the risk of developing sulfasalazine induced SJS/TEN (SJS/TEN vs. healthy control group: P=5.98×10⁻⁵, odds ratio=13.7 (3.6-52.4), sensitivity: 72.72%, specificity: 83.74%).

Further analysis of the HLA-B*1301 allele, HLA-B*3802 allele and HLA-B*3901 allele combination shows such combination significantly increases the correlation with and sensitivity in predicting the risk of developing sulfasalazine induced severe cutaneous adverse drug reactions (SCAR) (SCAR vs. healthy control group: P=3.21×10⁻⁸, odds ratio=7.6 (3.4-17.1), sensitivity: 68.75%, specificity: 78.32%).

Further analysis of the HLA-B*1301 allele, HLA-B*1502 allele, HLA-B*3802 allele and HLA-B*3901 allele combination shows such combination significantly increases the correlation with and sensitivity in predicting the risk of developing sulfasalazine induced severe skin adverse reactions (SCAR) (SCAR vs. healthy control group: P=2.67×10⁻⁷, odds ratio=7.1 (3.0-16.9), sensitivity: 75.00%, specificity: 60.35%).

Based on the above results, the presence of HLA-B*1301 allele, HLA-B*1502 allele, HLA-B*3802 allele, HLA-B*3901 allele, a HLA-B*1301 allele and HLA-B*3901 allele combination, a HLA-B*1502 allele and HLA- B*3802 allele combination, a HLA-B*1301 allele, HLA-B*3802 allele and HLA-B*3901 allele combination or a HLA-B*1301 allele, HLA-B*1502 allele, HLA-B*3802 allele and HLA-B*3901 allele combination can be used to assess the risk of developing adverse cutaneous drug reactions caused by disease-modifying antirheumatic drugs.

Table 1. Analysis and Comparison of the HLA-B*1301 and/or HLA-B*1502 and/or HLA-B*3802 and/or HLA-B*3901 genotype in 32 patients with severe cutaneous adverse drug reaction induced by the disease-modifying antirheumatic drug, sulfasalazine and 755 normal healthy controls.

| | | Health | Odds | | | |
|---|---|---|---|---|---|---|
| HLA-B | SCAR | Control | Ratio | P | Sensitivity | Specificity |
| and SCAR | [symbol] (%) | N (%) | (95% CI) | value | (%) | (%) |
| HLA-B*13:01 | | | | | | |
| Sulfasalazine- | 1/11 | 114/941 | 0.7 | 1 | 9.09 | 87.89 |
| SJS/TEN | (9.09%) | (12.11%) | (0.1 to 5.7) | | | |
| Sulfasalazine- | 8/21 | 114/941 | 4.5 | 2.59 × 10⁻³ | 38.10 | 87.89 |
| DRESS | (38.10%) | (12.11%) | (1.8 to 11.0) | | | |
| Sulfasalazine- | 9/32 | 114/941 | 2.8 | 0.014 | 28.13 | 87.89 |
| SCAR | (28.13%) | (12.11%) | (1.3 to 6.3) | | | |
| HLA-B*15:02 | | | | | | |
| Sulfasalazine- | 5/11 | 87/941 | 8.2 | 2.19 × 10⁻³ | 45.45 | 90.75 |
| SJS/TEN | (45.45%) | (9.25%) | (2.4 to 27.4) | | | |
| Sulfasalazine- | 2/21 | 87/941 | 1.0 | 1 | 9.53 | 90.75 |
| DRESS | (9.53%) | (9.25%) | (0.2 to 4.5) | | | |
| Sulfasalazine- | 7/32 | 87/941 | 2.7 | 0.028 | 21.88 | 90.75 |
| SCAR | (21.88%) | (9.25%) | (1.2 to 6.5) | | | |
| HLA-B*38:02 | | | | | | |
| Sulfasalazine- | 6/11 | 71/941 | 14.7 | 7.72 × 10⁻⁵ | 54.54 | 92.45 |
| SJS/TEN | (54.54%) | (7.55%) | (4.4 to 49.4) | | | |
| Sulfasalazine- | 0/21 | 71/941 | 0.3 | 0.394 | 0 | 92.45 |
| DRESS | (0%) | (7.55%) | (0.02 to 4.7) | | | |
| Sulfasalazine- | 6/32 | 71/941 | 2.8 | 0.034 | 18.75 | 92.45 |
| SCAR | (18.75%) | (7.55%) | (1.1 to 7.1) | | | |
| HLA-B*39:01 | | | | | | |
| Sulfasalazine- | 0/11 | 43/941 | 1.1 | 1 | 0 | 95.43 |
| SJS/TEN | (0%) | (4.57%) | (0.1-19.0) | | | |
| Sulfasalazine- | 8/21 | 43/941 | 12.2 | 4.30 × 10⁻⁶ | 38.10 | 95.43 |
| DRESS | (38.10%) | (4.57%) | (4.6-32.5) | | | |
| Sulfasalazine- | 9/32 | 43/941 | 8.2 | 1.93 × 10⁻⁵ | 28.13 | 95.43 |
| SCAR | (28.13%) | (4.57%) | (3.6 to 18.7) | | | |
| HLA-B*13:01/ | | | | | | |
| B*39:01 | | | | | | |
| Sulfasalazine- | 15/21 | 151/941 | 13.1 | 4.27 × 10⁻⁸ | 71.43 | 83.95 |
| DRESS | (71.43%) | (16.05%) | (5.0-34.2) | | | |
| Sulfasalazine- | 16/32 | 151/941 | 5.2 | 1.39 × 10⁻⁵ | 50.00 | 83.95 |
| SCAR | (50.00%) | (16.05%) | (2.6-10.7) | | | |
| HLA-B*15:02/ | | | | | | |
| B*38:02 | | | | | | |
| Sulfasalazine- | 8/11 | 153/941 | 13.7 | 5.98 × 10⁻⁵ | 72.72 | 83.74 |
| SJS/TEN | (72.72%) | (16.26%) | (3.6-52.4) | | | |
| Sulfasalazine- | 10/32 | 153/941 | 2.3 | 0.049 | 31.25 | 83.74 |
| SCAR | (31.25%) | (16.26%) | (1.1-5.0) | | | |
| HLA- | | | | | | |
| B*13:01/B*38:02/ | | | | | | |
| B*39:01 | | | | | | |
| Sulfasalazine- | 22/32 | 204/941 | 7.6 | 3.21 × 10⁻⁸ | 68.75 | 78.32 |
| SCAR | (68.75%) | (21.68%) | (3.4-17.1) | | | |
| HLA- | | | | | | |
| B*13:01/B*15:02/ | | | | | | |
| B*38:02/B*39:01 | | | | | | |
| Sulfasalazine- | 24/32 | 279/941 | 7.1 | 2.67 × 10⁻⁷ | 75.00 | 60.35 |
| SCAR | (75.00%) | (29.65%) | (3.0-16.9) | | | |

The foregoing is a description of the preferred embodiments of the present invention, and the present invention will be described in detail, and the subject matter of the present invention can be changed and modified without departing from the spirit and scope of the invention. Modifications are intended to be included within the scope of the following claims.

### SEQUENCE LISTING

The instant application contains a Sequence Listing which is being submitted in ASCII format via EFS-WEB and is hereby incorporated by reference in its entirety.

File name: 14 SQL

Creation date: Oct. 18, 2021

Byte size: 3,440 bytes

1-12. (canceled)

13. A method for assessing the risk of developing a severe cutaneous adverse drug reactions caused by a disease-modifying antirheumatic drug and treating said severe cutaneous adverse drug reaction in a subject, comprising the following steps:
(a) detecting at least one of the following alleles from a sample from the subject: HLA-B*1502 allele, HLA-B*3802 allele or a combination of HLA-B*1301 allele and HLA-B*3901 allele;
(b) the presence of at least one of the following alleles from the sample from the subject:
HLA-B*1502 allele, HLA-B* 3802 allele or the combination of HLA-B*1301 allele and HLA-B*3901 allele, indicates the subject has a risk of developing the severe cutaneous adverse drug reaction induced by the disease-modifying antirheumatic drug compared to a patient without the corresponding HLA allele; and thereafter
(c) based on the detecting the presence of at least one of the following alleles from the sample from the subject: HLA-B*1502 allele, HLA-B* 3802 allele or the combination of HLA-B*1301 allele and HLA-B*3901 allele, administering a drug to treat the severe cutaneous adverse drug reaction.

14. The method according to claim 13, wherein said severe cutaneous adverse drug reaction comprises at least one adverse reaction selected from the following: Stevens Johnsons Syndrome (SJS), toxic epidermal necrolysis (TEN) or drug rash with eosinophilia and systemic symptoms (DRESS).

15. The method according to claim 13, wherein said HLA-B*1502 allele, HLA-B*3802 allele or a combination of HLA-B*1301 allele and HLA-B*3901 allele are detected in the DNA, RNA, proteins, cells or serum sample prepared from the peripheral blood of the subject.

16. The method according to claim 13, wherein said disease-modifying antirheumatic drug is Sulfasalazine, Mesalazine, Sulfapyridine or Olsalazine.

17. The method according to claim 13, wherein the drug to treat the severe cutaneous adverse drug reaction is liquid, steroid, immunoglobulin, cyclosporine, anti-TNF-α agent or plasma replacement.

18. A method for assessing the risk of developing a severe cutaneous adverse drug reaction induced by a disease-modifying antirheumatic drug and reducing the incidence of said severe cutaneous adverse drug reaction in a subject, comprising the following steps:
(a) detecting at least one of the following alleles from a sample from the subject : HLA-B*1502 allele, HLA-B*3802 allele or a combination of HLA-B*1301 allele and HLA-B*3901 allele;
(b) the presence of at least one of the following alleles from the sample from the subject:
HLA-B*1502 allele, HLA-B* 3802 allele or the combination of HLA-B*1301 allele and HLA-B*3901 allele, indicates that the subject has an increased risk of developing the severe cutaneous adverse drug reaction compared to a patient without the corresponding HLA allele; and
(c) based on the detecting the presence of at least one of the following alleles from the sample from the subject: HLA-B*1502 allele, HLA-B* 3802 allele or the combination of HLA-B*1301 allele and HLA-B*3901 allele, the subject is not given the disease-modulating anti-rheumatic drug.

19. The method according to claim 18, wherein said severe cutaneous adverse drug reaction comprises at least one adverse reaction selected from the following: Stevens Johnsons Syndrome (SJS), toxic epidermal necrolysis (TEN) or drug rash with eosinophilia and systemic symptoms (DRESS).

20. The method according to claim 18, wherein said HLA-B*1502 allele, HLA-B*3802 allele or the combination of HLA-B*1301 allele and HLA-B*3901 allele are detected in the DNA, RNA, proteins, cells or serum sample prepared from the peripheral blood of the subject.

21. The method according to claim 18, wherein said disease-modifying antirheumatic drug is Sulfasalazine, Mesalazine, Sulfapyridine or Olsalazine.
